(19) United States
(12) Patent Application Publication
(10) Pub. No.: US 2026/0144429 A1
(43) Pub. Date: May 28, 2026

(54) DEV ICES, SYSTEMS, AND METHODS FOR SEPARATING TISSUE

(71) Applicant: Boston Scientific Scimed, Inc., Maple Grove, MN (US)

(72) Inventors: Timothy Noah Foster-Frau, Austin, TX (US); Gregory Todd Dalton, Austin, TX (US); Alexander R. Cohen, Austin, TX (US); Ryan Davis Gilkey, Austin, TX (US)

(73) Assignee: Boston Scientific Scimed, Inc., Maple Grove, MN (US)

(21) Appl. No.: 19/400,791

(22) Filed: Nov. 25, 2025

Related U.S. Application Data
(60) Provisional application No. 63/725,130, filed on Nov. 26, 2024.

(51) Int. Cl. A61B 1/00

(52) U.S. Cl. CPC A61B 1/00087

(57) ABSTRACT

A tissue separator device having a first tissue separator arm and a second tissue separator arm extending distally from a proximal mounting portion configured to be mounted on the distal end of a medical delivery device. The medical delivery device may be a medical scope, the tissue separator device leaving the distal end of the medical scope unimpeded as the tissue separator arms are actuated to separate tissue. The tissue separator arms may engage tissue to inhibit slippage with respect to tissue and/or to stabilize the medical delivery device with respect to tissue.

### CROSS-REFERENCE TO RELATED APPLICATIONS

This application claims the benefit of priority under 35 U.S.C. § 119 to U.S. Provisional Application No. 63/725,130, filed Nov. 26, 2024, the entire disclosure of which is hereby incorporated by reference herein for all purposes.

### FIELD

The present disclosure relates generally to the field of medical devices, systems, and methods for separating tissue. More particularly, the present disclosure relates to medical devices, systems, and methods using tissue separators which are movable with respect to each other to mechanically separate or dissect tissue.

### BACKGROUND

Various procedures involve cutting, dissecting, resecting, excising, etc., anatomical structures (e.g., biological tissue, including lesions, etc.). One such procedure is third space endoscopy (also known as submucosal endoscopy), which may access deeper layers of tissue within the body (e.g., the gastrointestinal (GI) tract) by tunneling into the tissue, such as between structurally differentiated layers of tissue. For instance, in the GI system, tunneling may be performed in the submucosal space with an endoscope, without compromising the integrity of the overlying mucosa. Typically, a fluid (e.g., water, saline, air, etc.) is injected at the treatment site to elevate/lift the tissue (to create a “bleb”) to facilitate cutting of the tissue, such as with a knife. For instance, a lifting agent may be injected into the submucosal layer to separate the mucosal layer from the muscularis layer. A sharp cutting instrument such as a blade, a knife, an electrosurgical knife, etc., may then be used to create a small incision. The sharp cutting instrument, or a blunt dissector may then be inserted to cut through the third space between tissue layers. Creating space and traction for a cutting device generally improves the speed and safety of the procedure. For instance, during procedures such as endoscopic submucosal dissection (“ESD”), increasing the margin between the cutting surface of a cutting device and the muscularis tissue layer in the gastrointestinal (“GI”) tract (e.g., in the stomach, the intestines, etc.) allows safer dissection and removal of target tissue (e.g., a lesion). Often, once a cut has been made, and access to the third space achieved, advancing a tool further into the third space and against tissue, to cut and/or to separate tissue, typically results in the tool to rebound or be pushed back out of the space. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a device for separating tissue extends along a longitudinal axis from a proximal end to a distal end, and includes a proximal mounting portion along the proximal end of the device and defining a counterbore sized, shaped, configured, and/or dimensioned to receive a distal end of a medical delivery device; a first tissue separator arm extending distally from the proximal mounting portion and movable laterally away from the longitudinal axis; and a second tissue separator arm extending distally from the proximal mounting portion and movable laterally away from the longitudinal axis, the first tissue separator arm and the second tissue separator arm being configured to contact and separate tissue upon moving laterally away from the longitudinal axis.

In some aspects, the first tissue separator arm and the second tissue separator arm are configured to move laterally away from the longitudinal axis as well as proximally to separate tissue as well as to push tissue proximally.

Additionally or alternatively, the first tissue separator arm and the second tissue separator arm are pivotable with respect to the proximal mounting portion.

Additionally or alternatively,, the first tissue separator arm and the second tissue separator arm are capable of flexing to form a concave tissue engaging surface. Additionally or alternatively, the first tissue separator arm and the second tissue separator arm each include a plurality of axially spaced apart articulation sections configured to facilitate flexing of the first tissue separator arm and the second tissue separator arm to form concave tissue engaging surfaces. Additionally or alternatively, the device further includes a tissue engagement feature along the tissue engaging surface configured to engage tissue to prevent slippage of the first tissue separator arm and the second tissue separator arm with respect to tissue contacted by the tissue engaging surfaces.

Additionally or alternatively, the first tissue separator arm and the second tissue separator arm are each formed of a section of a tubular element and are separated by slits.

Additionally or alternatively, proximal regions of the first tissue separator arm and the second tissue separator arm are thinner than distal regions of the first tissue separator arm and the second tissue separator arm to allow flexing of the first tissue separator arm and the second tissue separator arm with respect to the proximal mounting portion.

Additionally or alternatively, wherein the first tissue separator arm and the second tissue separator arm are each formed of wires forming a peripheral frame without material extending across the full width of the first tissue separator arm and the second tissue separator arm.

In some aspects, the first tissue separator arm and the second tissue separator arm are expandable. Additionally or alternatively, the first tissue separator arm and the second tissue separator arm are inflatable.

In some aspects, the first tissue separator arm and the second tissue separator arm form a tapered distal end of the device.

In accordance with various principles of the present disclosure, an end cap is configured for operable coupling with a medical scope having a viewing device, the end cap extending along a longitudinal axis from a proximal end to a distal end and having a proximal mounting portion along the proximal end of the end cap and defining a counterbore sized, shaped, configured, and/or dimensioned to receive a distal end of a medical scope; a first tissue separator arm extending distally from the proximal mounting portion and movable laterally away from the longitudinal axis; and a second tissue separator arm extending distally from the proximal mounting portion and movable laterally away from the longitudinal axis, the first tissue separator arm and the second tissue separator arm being configured to contact and separate tissue upon moving laterally away from the longitudinal axis.

In some aspects, the first tissue separator arm and the second tissue separator arm are spaced apart within the end cap to leave a distal end of a medical scope inserted into the proximal mounting portion unimpeded during a procedure.

Additionally or alternatively, at least one of the first tissue separator arm or the second tissue separator arm includes a tissue engagement feature configured to prevent slippage of the first tissue separator arm and the second tissue separator arm with respect to tissue contacted by the tissue engagement feature to stabilize the medical scope with respect to a target area.

In accordance with various principles of the present disclosure, a method of separating tissue includes creating an initial incision in tissue at a target area; inserting a distal end of a tissue separator device mounted on a distal end of a medical delivery device into the incision; distally advancing the distal end of the tissue separator device into the incision; and moving a first tissue separator arm and a second tissue separator arm of the tissue separator device laterally apart from each other to separate tissue at the incision.

In some aspects, the method further includes moving the first tissue separator arm and the second tissue separator arm proximally to push tissue proximally and to advance the medical delivery device distally into the target area.

Additionally or alternatively, the method further includes engaging a tissue engagement feature extending from at least one of the first tissue separator arm or the second tissue separator arm into tissue to inhibit slippage of the tissue separator device with respect to tissue.

Additionally or alternatively, the method further includes engaging a tissue engagement feature extending from at least one of the first tissue separator arm or the second tissue separator arm into tissue to stabilize the medical delivery device with respect to tissue.\

In some aspects, the medical delivery device is a medical scope, the method further including visualizing, with the medical scope, tissue being separated by the first tissue separator arm and the second tissue separator arm extending distal to the distal end of the medical scope.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an example of an embodiment of a tissue separating system formed in accordance with aspects of the present disclosure with the tissue separator device thereof illustrated in a first configuration and also in a detail view.

FIG. 1A illustrates an example of an embodiment of a tissue separating system similar to the tissue-separating system illustrated in FIG. 1, but with an alternate configuration of portions of the tissue separator device.

FIG. 2 illustrates a tissue separator device such as illustrated in FIG. 1 or FIG. 1A, but in a second configuration.

FIG. 3 illustrates an example of an embodiment of a tissue separating system formed in accordance with various principles of the present disclosure with the tissue separator device thereof illustrated in a first configuration and also in a detail view.

FIG. 4 illustrates a tissue separator device such as illustrated in FIG. 3, but in a second configuration.

FIG. 5 illustrates an example of an embodiment of a tissue separating system formed in accordance with various principles of the present disclosure with the tissue separator device thereof illustrated in a first configuration and also in a detail view.

FIG. 6 illustrates a tissue separator device such as illustrated in FIG. 5, but in a second configuration.

FIG. 7 illustrates a tissue separator device such as illustrated in FIG. 5 and FIG. 6, but in a third configuration.

FIG. 8 illustrates an example of an embodiment of a tissue separating system formed in accordance with various principles of the present disclosure with the tissue separator device thereof illustrated in a first configuration as well as in a cross-sectional detail view.

FIG. 9 illustrates a tissue separator device such as illustrated in FIG. 8, but in a second configuration.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, a tissue separator device is configured to be mounted on a distal end of a medical delivery device to separate tissue at a treatment site. In some aspects, the tissue separator device may thus be considered an endcap. The tissue separator device may be formed as a specialized attachment for a medical delivery device configured and designed to allow for controlled separation of tissue and optionally also to allow for secure anchoring of the medical delivery device during minimally invasive procedures. It will be appreciated that terms such as target site, target area, target tissue site, target tissue area, target area of tissue, target treatment area, treatment area, target treatment site, treatment site, etc., may be used interchangeably herein, without intent to limit, to refer to an area or region of tissue within which a target tissue is located. The term target tissue (and other variations thereof, such as treatment tissue) is used herein to refer to the tissue with respect to which a procedure is to be performed or which is to be treated or otherwise operated on or affected by the devices and/or systems and/or methods disclosed herein. Thus, the target site may be understood as an area or region extending outwardly from or around or surrounding the target tissue (specific tissue in the target tissue area), such as the region a medical professional would consider a working area around the target tissue for performing a procedure with respect to the target tissue.

In some aspects, the medical delivery device is configured to deliver another tool to be inserted into the space created by the tissue separator device. It will be appreciated that terms such as devices, instruments, tools, components, accessories, etc., may be used interchangeably herein without intent to limit unless otherwise indicated. In some aspects, the medical delivery device may be a medical scope, such as an endoscope. In some aspects, the tissue separator device is designed to enhance tissue manipulation, such as by a tool delivered by the medical delivery device, and/or scope stabilization. Mounting of a tissue separator device on the end of the medical delivery device allows the tissue separator device to be fixed in place with respect to the medical delivery device so that medical tools can be used with more force than previously achievable.

In some aspects, a tissue separator device mounted on a distal end of a medical delivery device may be configured with a blunt distal end to perform a blunt tissue dissection. As noted above, a potential problem with manipulating tissue, such as with a blunt dissector, is rebound of the medical delivery device from the treatment site, such as a result of rebounding upon encountering tissue. A tissue separator device is formed in accordance with various principles of the present disclosure to resist backing out of tissue during operation. For instance, in some aspects, a tissue separator device formed in accordance with various principles of the present disclosure has separator arms which open substantially symmetrically, with substantially symmetrical opposing forces. Additionally or alternatively, the separator arms open laterally apart from each other and then move proximally to move the tissue separator device further distally into tissue to further separate tissue. Additionally or alternatively, one or both separator arms includes a tissue engagement structure configured to engage with tissue to further inhibit backout of the tissue separator device from the treatment site.

In some aspects, the medical delivery device includes a viewing device, such as a camera. Positioning of a tissue separator device on the distal end of the medical delivery device allows tissue separation to be performed in front of the medical delivery device camera, allowing improved viewing of a procedure. A medical professional may thus be able to respond more quickly than with other devices if a bleed is observed during the procedure.

Various embodiments of devices, systems, and methods for separating tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

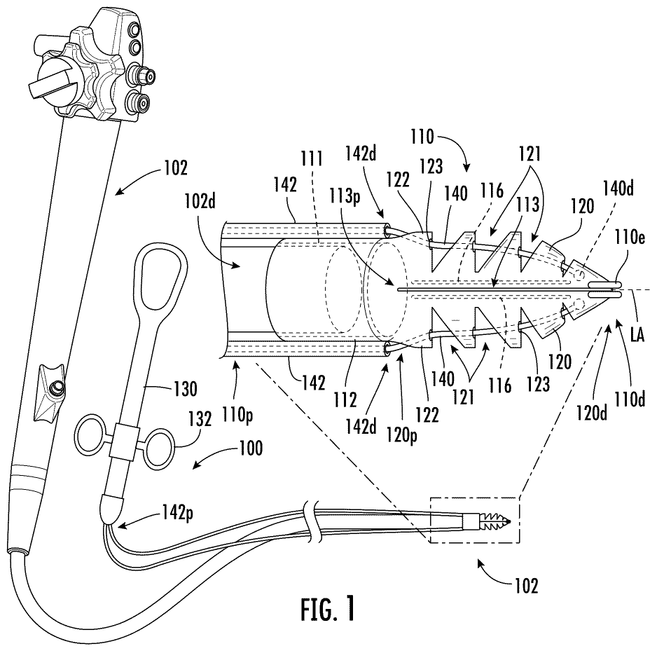

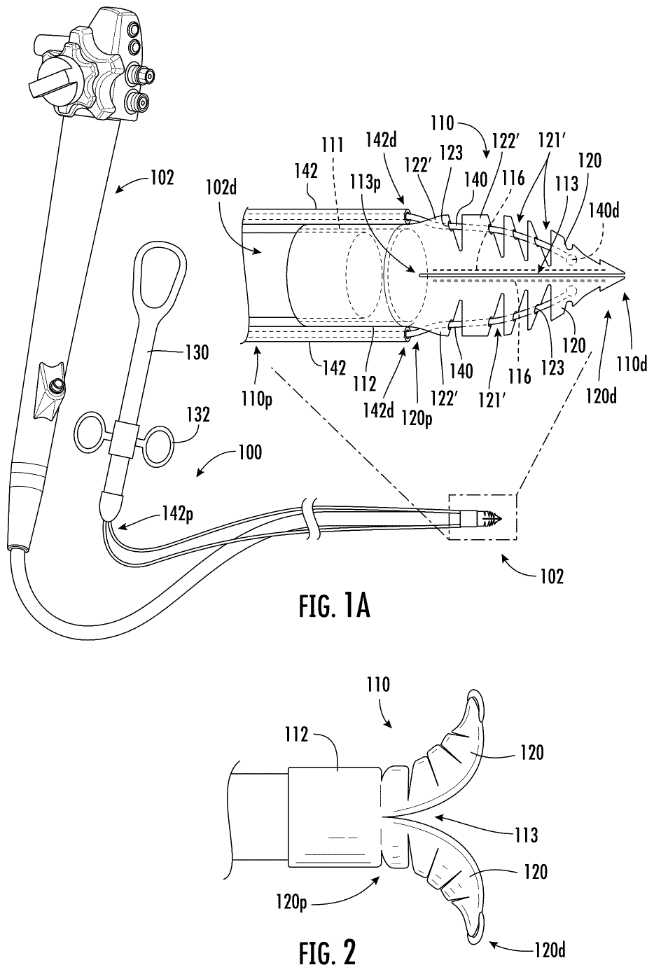

Turning now to the drawings, a tissue separating system 100 formed in accordance with various principles of the present disclosure is illustrated in FIG. 1. The illustrated tissue-separating system 100 includes a tissue separator device 110 along a distal end 100d of the tissue-separating system 100. The tissue separator device 110 is operably associated with a medical delivery device 102, which may be considered a part of the tissue-separating system 100 or a device in conjunction with which the tissue-separating system 100 is used. In some aspects, the proximal end 110p of the tissue separator device 110 includes a counterbore 111 defined in a proximal mounting portion 112 of the tissue separator device 110. The counterbore 111 is configured to receive the distal end 100d of the medical delivery device 102. The counterbore 111 may ensure secure mounting and attachment of the tissue separator device 110 to the medical delivery device 102 without blocking the distal end 102d of the medical delivery device 102. In some aspects, the counterbore 111 extends the entire length of the proximal mounting portion 112 of the tissue separator device 110, forming a lumen through the tissue separator device 110. It will be appreciated that the inner diameter of the lumen defined by the counterbore 111 need not be constant. Preferably, the functionality of the medical delivery device 102 is not obstructed or impeded by the structure and position of the tissue separator device 110 with respect to the distal end 102d of the medical delivery device 102. For instance, the counterbore 111 and optional longitudinally-extending lumen defined by the counterbore 111 along the length of the proximal mounting portion 112 of the tissue separator device 110 allows various functions, such as delivery of a medical device through a working channel of the medical delivery device 102, observation of the procedure using a viewing device of the medical delivery device 102, irrigation/aspiration of the treatment site by the medical delivery device 102, illumination of the treatment site by a lighting element the medical delivery device 102, etc. In some aspects, the tissue separator device 110 is further secured with an additional securing element, such as adhesive, tape, a band, a clip, etc., the present disclosure not being limited in this regard.

In accordance with various principles of the present disclosure, the tissue separator device 110 includes at least two tissue separator arms 120 movable between a delivery configuration (such as illustrated in FIG. 1 and FIG. 1A) and a tissue-separating configuration (such as illustrated in FIG. 2). In the delivery configuration, the tissue separator arms 120 extend generally alongside and generally adjacent each other, such as illustrated in FIG. 1. An alternate embodiment of the tissue separator device 110 is illustrated in a delivery configuration in FIG. 1A. The embodiments of FIG. 1 and FIG. 1A differ in the configurations of features facilitating shifting to a tissue-separating configuration, as discussed in further detail below. In some aspects, the tissue separator arms 120 may be in a relatively compact configuration widthwise (radially and generally orthogonal with respect to the longitudinal axis LA), at least relative to other configurations of the tissue separator arms 120, when the tissue separator arms 120 are in the delivery configuration. In some aspects, the compact configuration facilitates delivery (e.g., transluminal delivery) of the tissue separator device 110 to a treatment site within a patient's body. In some aspects, the tissue separator arms 120 may be adjacent each other, when in the delivery configuration, to facilitate insertion into tissue (e.g., through an incision made by another device, such as a sharper cutting instrument). In some aspects, the tissue separator arms 120 are inserted together into tissue, such as without intentionally extending tissue between the adjacent tissue separator arms 120. In some aspects, the tissue separator arms 120 define a tapered distal end 110d of the tissue separator device 110. For instance, each of the tissue separator arms 120 may extend distally away from the proximal mounting portion 112 along the proximal end 110p of the tissue separator device 110, and may additionally be inclined toward the other of the tissue separator arms 120 and toward the longitudinal axis LA of the tissue separator device 110. Such tapered distal end 110d of the tissue separator device 110 facilitates insertion of the tissue separator device 110 into small incisions which may be created by a sharper instrument so that blunt dissection may be performed as the primary manner of separating tissue at a treatment site. In some aspects, the tapered distal end 110d of the tissue separator device 110 provides a blunt surface to tissue prior to operation of the tissue separator device 110 to separate tissue. In some aspects, the tapered distal end 110d of the tissue separator device 110 is generally conical with a blunt distalmost end 110e which need not be sharp enough to cut tissue.

In the tissue-separating configuration, such as illustrated in FIG. 2, the tissue separator arms 120 are moved apart from each other, such as to move apart tissue surrounding the tissue separator arms 120 when the tissue separator arms 120 are in the delivery configuration. Separation of the tissue separator arms 120 of the tissue separator device 110 such as illustrated in FIG. 2 widens the incision in the tissue into which the distal end 110d of the tissue separator device 110 is initially inserted. The distal end 110d of the tissue separator device 110 is formed with a slit 113 separating the tissue separator arms 120. As may be appreciated, the slit 113 does not extend through the entire length of the tissue separator device 110. Instead, the proximal end 113p of the slit 113 ends at or adjacent the proximal mounting portion 112 of the tissue separator device 110 (e.g., closer to the proximal end 120p of the tissue separator arms 120 than the distal end 120d of the tissue separator arms 120). It will be appreciated that the tissue separator arms 120, when in a tissue-separating configuration such as illustrated in FIG. 2, provide an open, unobstructed, unimpeded area in front of (distal to) the distal end 102d of the medical delivery device 102 so as to allow various functions of the medical delivery device 102 to be performed without interference by the tissue separator arms 120.

In some aspects, the tissue separator arms 120 of the tissue separator device 110 are formed substantially symmetrically on either side of a plane of symmetry through which the longitudinal axis LA of the tissue separator device 110 extends. For instance, the tissue separator device 110 may be considered to be formed with a blunt, tapered distal end 110d with the slit 113 extending within (or parallel to) the plane of symmetry, proximally from the distal end 110d of the tissue separator device 110 and toward the proximal end 110p of the tissue separator device 110. Substantially symmetrical formation of the tissue separator arms 120 may allow for substantially symmetrical opening of the tissue separator arms 120 and resulting symmetrical forces imparted by the tissue separator arms 120 on the tissue to be separated.

In some aspects, the tissue separator device 110 is constructed from a flexible material which allows pivoting of the tissue separator arms 120, yet provides sufficient rigidity to the tissue separator arms 120 to push against tissue engaged by the tissue separator arms 120. More specifically, as the tissue separator arms 120 separate from each other and move proximally, the tissue separator arms 120 push off the tissue the tissue separator arms 120 contact. Separation of the tissue separator arms 120 thus not only separates tissue (as the tissue separator arms 120 move laterally apart), but may also further advances the tissue separator device 110 distally into tissue at the treatment site (as the tissue separator arms 120 move proximally, pushing off tissue and thus advancing distally). The tissue separator device 110 may be formed from any suitable medical grade material, such as a material comparable to silicone but softer than a polymer, such as acrylonitrile butadiene styrene (ABS). In some aspects, the tissue separator device 110 can be manufactured using 3D printing techniques with elastic resin materials. An optional stiffener wire or internal loop 116 may be provided in or along one or both of the tissue separator arms 120 to fine-tune the rigidity/flexibility of the tissue separator arms 120, such as to fine tune the force and articulation characteristics of the tissue separator arm 120. In some aspects, the stiffener wire 116 extends along the inner surface of the tissue separator arms 120. Additionally or alternatively, the stiffener wire 116 does not extend proximally or distally past the tissue separator arms 120.

In some aspects, outward pivoting of the tissue separator arms 120 is accompanied by flexing and bending of the tissue separator arms 120. As the tissue separator arms 120 flex, bend, deform, etc., they may become concavely curved along the exterior and convexly curved along the interior surface (e.g., the surface formed along or by the slit 113). To facilitate such curving (and optionally to minimize any internal stress or strain on the material), the tissue separator arms 120 of the examples of tissue separator devices 110 illustrated in FIG. 1, FIG. 1A, and FIG. 2 may be formed with longitudinally spaced apart gaps 121, 121′ along the exterior surface thereof (directed away from the longitudinal axis LA to engage tissue at the treatment site). The gaps 121, 121′ extend transverse to the longitudinal axis LA of the tissue separator device 110 (i.e., at an angle other than 0° or 180° with respect to the longitudinal axis LA) and are sized, shaped, configured, positioned, and/or dimensioned to ensure flexing and optional deformation of the tissue separator arms 120 in the desired outward direction upon actuation (described in further detail below). In some aspects, the gaps 121, 121′ may be considered to form axially spaced apart articulation sections 122, 122′ of the tissue separator arms 120. In some aspects, the gaps 121, 121′ form a wedge-shaped space between the articulation sections 122, 122′ to facilitate flexing of the tissue separator arms 120, such as to form concave tissue-engaging surfaces. For instance, the gaps 121, 121′ may be configured so that the longitudinal distance between the spaced apart articulation sections 122, 122′ increases in a direction from a radially-inward position of the gap 121, 121′ to a radially-outward position of the gaps 121, 121′ (i.e., in a radially outwardly direction from the longitudinal axis LA). In the examples of embodiments illustrated in FIG. 1 and FIG. 1A, the wedge-shaped gaps 121, 121′ are formed between a wall of a first articulation section 122, 122′ which is generally perpendicular to the longitudinal axis LA, and a wall of an adjacent articulation section 122, 122′ which is at an angle (other than 0° or 180°) with respect to the longitudinal axis LA. In the example of an embodiment illustrated in FIG. 1, the wall forming the proximal side of the wedge-shaped gap 121 is perpendicular to the longitudinal axis LA, and the wall forming the distal side of the wedge-shaped gap 121 is at an angle (other than 0° or 180°) with respect to the longitudinal axis LA. In contrast, in the example of an embodiment illustrated in FIG. 1A, the wall forming the proximal side of the wedge-shaped gap 121 is at an angle (other than 0° or 180°) with respect to the longitudinal axis LA, and the wall forming the distal side of the wedge-shaped gap 121 is perpendicular to the longitudinal axis LA. It will be appreciated that other configurations of gaps 121, 121′ and/or articulation section 122, 122′ are within the scope and spirit of the present disclosure.

In some aspects, articulation sections 122, 122′ engage tissue as the tissue separator arms 120 are moved apart. As the tissue separator arms 120 continue to flex, tissue may be caught in the gaps 121, 121′ between the articulation sections 122, 122′. The tissue separator arms 120 may thereby be engaged and secured to tissue to resist movement of the tissue separator device 110 with respect to the tissue. As such, outward and proximal flexing of the tissue separator arms 120 engage and move tissue apart and proximally, thereby advancing the tissue separator device 110 distally. The tissue separator arms 120 may then be returned to a closed configuration and then reopened to the tissue-separating configuration to further separate tissue and further distally advance into the tissue. As may be appreciated, such engagement of tissue in the gaps 121, 121′ between the articulation sections 122, 122′ secures the tissue separator device 110 with respect to the tissue to resist any counterforces as the tissue separator device 110 is pushed distally against tissue. The gaps 121, 121′ and the articulation sections 122, 122′ may thus be considered an anti-backout feature of the tissue separator device 110 inhibiting or preventing the medical delivery device 102 from being pushed proximally and backed out of the treatment site during tissue separation performed with the tissue separator device 110 (such as in response to pushing against tissue).

In some aspects, as illustrated in FIG. 1A, the distal end 120d of one or both of the tissue separator arms 120 of the tissue separator device 110 may include a tissue engagement feature 124 extending radially outwardly from the exterior of the tissue separator device 110. The tissue engagement feature 124 is sized, shaped, configured, positioned, and/or dimensioned to engage tissue. More particularly the tissue engagement feature 124 may be sized, shaped, configured, positioned, and/or dimensioned to grasp and/or penetrate tissue to grip tissue and to prevent slippage of the tissue separator arms 120 with respect to the tissue. For instance, the tissue engagement feature 124 may be in the form of an aggressive edge designed to grip tissue effectively. When the tissue separator arms 120 are shifted into a tissue-separating configuration, a tissue engagement feature 124 on one or both tissue separator arms 120 engages and optionally penetrates into tissue, allowing the tissue separator arms 120 to clamp onto surrounding tissue to fix the tissue-separating system 100 in place with respect to tissue at the treatment site. The tissue engagement features 124 may thus be considered to be anti-backout features which inhibit or prevent the tissue-separating system 100 from being pushed proximally and backed out of the treatment site during tissue separation (such as in response to pushing against tissue).

Actuation of the tissue separator arms 120 may be effected in any of a variety of manners known to those of ordinary skill in the art. In the example of an embodiment illustrated in FIG. 1, a control handle 130 is provided at the proximal end 100p of the tissue-separating system 100. At least one longitudinally translatable control wire 140 extends from the control handle 130 to the tissue separator device 110 to actuate the tissue separator arms 120 to shift between the closed configuration and an open, tissue-separating configuration. For instance, a proximal end 140p of the control wire 140 may be operably coupled to an actuator 132 of the control handle 130 and a distal end 140d of the control wire 140 may be fixed with respect to a tissue separator arm 120. Movement of the control wire 140, such as by actuation of the actuator 132 to longitudinally translate the control wire 140, shifts the tissue separator arm 120 between a closed configuration and a tissue-separating configuration. For instance, the control wire 140 may be fixed with respect to the distal end 120d of the tissue separator arm 120 to pull the distal end 120d of the tissue separator arm 120 proximally, such as with respect to the proximal mounting portion 112 and/or the control handle 130, to move proximally along the longitudinal axis LA as well as laterally away from the longitudinal axis LA of the tissue separator device 110. The tissue separator arms 120 thereby shift from a closed configuration to a tissue-separating configuration. The tissue separator arms 120 may be biased to return to a closed configuration upon release of proximal pulling force on the control wires 140 and/or may be returned to a closed configuration by distal longitudinal translation of the control wires 140 (e.g., by pushing the control wires 140 distally).

In the example of an embodiment illustrated in FIG. 1, the control wire 140 slidably extends through a sheath 142 from the control handle 130 to the tissue separator arm 120. In some aspects, the sheath 142 is a flexible tubular elongate member, such as a coil catheter, capable of extending through curved or tortuous pathways within the patient's body to reach a treatment site within the patient's body. In some aspects, the sheath 142 provides rotational control and/or a spring-like return action and/or sufficient hoop strength to facilitate navigation with a patient's body. The proximal end 142p of the sheath 142 may be fixedly coupled with respect to the control handle 130, and at least the distal end 142d of the sheath 142 may be fixedly coupled with respect to the tissue separator device 110, such as with respect to the proximal mounting portion 112. In the example of an embodiment illustrated in FIG. 1, FIG. 1A, and FIG. 2, the control wire 140 extends out the distal end 142d of the sheath 142 and through control wire holes 123 defined through the tissue separator device 110. The distal end 140d of the control wire 140 is securely attached to the tissue separator arm 120, such as along the distal end 120d of the tissue separator arm 120, to prevent pull-through of the control wire 140. For instance, the control wire 140 may be secured with an increased diameter at the distal end 140d thereof (e.g., a knot, a crimp, or an attachment wider than the diameter of the control wire 140), and/or may be attached, such as by glue or a weld, to the tissue separator arm 120. In some aspects, the control wire holes 123 extend generally longitudinally through the proximal mounting portion 112. In some aspects, the control wire holes 123 may follow a radially-inwardly inclined path (towards the longitudinal axis LA) through the tissue separator arms 120, such as through the articulation sections 122, from a proximal end 120p of the tissue separator arm 120 to the distal end 120d of the tissue separator arm 120. Such inclined path may facilitate laterally outward movement of the tissue separator arm 120 upon proximal pulling of the control wire 140 along such inclined path.

As noted above, the proximal end 140p of the control wire 140 is operably coupled with an actuator 132 of the control handle 130, and moves with movement (e.g., sliding movement) of the actuator 132. Thus, movement of the actuator 132 moves the control wire 140 with respect to the sheath 142 (which has a proximal end 142p fixed with respect to the control handle 130) and with respect to the proximal mounting portion 112 of the tissue-separator actuator 132. Movement of the control wire 140 moves and/or articulates the tissue separator arm 120 (to which the distal end 140d of the control wire 140 is coupled) with respect to the sheath 142 and the proximal mounting portion 112 to shift the tissue separator arm 120 between the closed configuration and the tissue-separating configuration. In the illustrated example of an embodiment, the tissue separator device 110 utilizes a dual-wire control system, with a control wire 140 for each tissue separator arm 120. Each of the control wires 140 may be operated to shift a configuration of an associated tissue separator arm 120 as described above. In some aspects, pushing one control wire 140 and pulling the other control wire 140 may articulate the tissue separator device 110 in a controlled manner, such as to do a little “dance”, moving the tissue separator arms 120 laterally back and forth in opposite directions. Such articulation of the tissue separator arms 120 of the tissue separator device 110 may be useful in foreign object removal from a treatment site. The foreign object may be loosened by such movement of the tissue separator arms 120 and then retained within the negative space between the tissue separator arms 120 for removal from the treatment site.

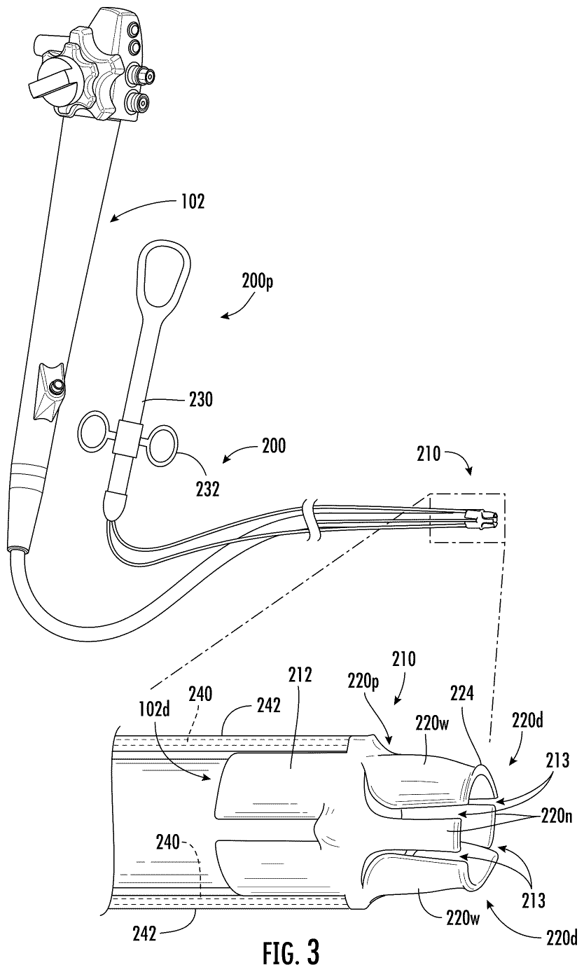

Various modifications may be made to a tissue separator system such as described above and in accordance with various principles of the present disclosure. For instance, in the example of an embodiment of a tissue-separating system 200 illustrated in FIG. 3, a tissue separator device 210 includes two or more tissue separator arms 220. In some aspects, the tissue separator arms 220 are formed from a generally tubular element, such as an end cap for a medical scope. In some aspects, the end cap includes a proximal mounting portion 212 mounted over the distal end 102d of the medical delivery device 102. The mounting of the proximal mounting portion 212 to the medical delivery device 102 may be similar to the mounting of the above-described proximal mounting portion 112, reference being made thereto, for the sake of brevity and without intent to limit, as applicable mutatis mutandis. In some aspects, proximal mounting portion 212 and the tissue separator arms 220, when mounted on the medical delivery device 102, leave an open internal area distal to the distal end 102d of the medical delivery device 102 so that devices extending from the distal end 102d of the medical delivery device 102 are capable of being used unimpeded and unobstructed by the tissue separator device 410. The material of a typical end cap (e.g., polypropylene) for a medical scope typically has sufficient rigidity to push tissue without being deformed, and thus may be suitable for a tissue separator device 210 formed in accordance with various principles of the present disclosure. In some aspects, proximal regions 220p of the tissue separator arms 220 may be thinner than distal regions to facilitate flexing or pivoting of the tissue separator arms 220 with respect to the proximal mounting portion 212 of the tissue separator device 210, leaving the distal ends 220d of the tissue separator arms 220 sufficiently strong to push tissue without deforming. The distal end 220d of one or more of the tissue separator arms 220 of the tissue separator device 210 may include a tissue engagement feature 224 extending radially outwardly from the exterior of the tissue separator device 210 and configured to engage tissue to inhibit movement of the tissue separator device 210 with respect to the engaged tissue. The tissue engagement feature 224 may be similar to the above-described tissue engagement features 124, and, for the sake of brevity and without intent to limit, reference is thus made to the above description of the tissue engagement features 124 as applicable to the tissue engagement features 224.

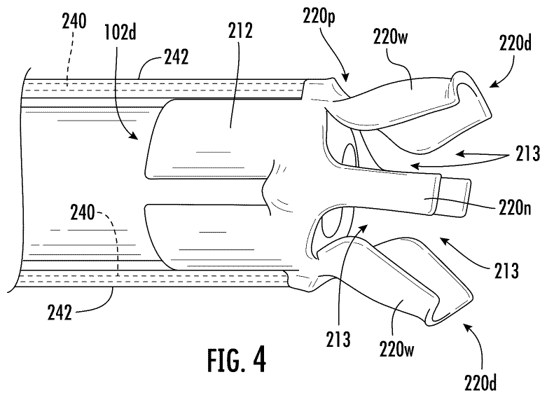

A plurality of generally longitudinal slits 213 are formed longitudinally along the tissue separator device 210 to define the tissue separator arms 220. In the example of an embodiment illustrated in FIG. 3, four tissue separator arms 220 are formed by four slits 213. The slits 213 may be equidistantly spaced apart, or at different circumferential distances. In the example of an embodiment illustrated in FIG. 3 and FIG. 4, the slits 213 are spaced apart to form two narrower tissue separator arms 220n and two wider tissue separator arms 220w. It will be appreciated that the present disclosure need not be limited to the illustrated configuration and other configurations are within the scope and spirit of the present disclosure.

Each of the tissue separator arms 220 has an associated control wire 240 which may extend from an actuator 232 of a control handle 230 at a proximal end 200p of the tissue-separating system 200 to the tissue separator arms 220. In some aspects, each control wire 240 extends to the distal end 220d of a respective tissue separator arm 220 and is fixedly coupled thereto. Proximal movement (e.g., proximal longitudinal translation) of the control wire 240 thus effects proximal and radially-outwardly lateral movement of the associated tissue separator arm 220 to shift the tissue separator arms 220 from a closed configuration to an open, tissue-separating configuration. The tissue separator arms 220 may be biased to return to a closed configuration upon release of proximal pulling force on the control wires 240 and/or may be returned to a closed configuration by distal longitudinal translation of the control wires 240 (e.g., by pushing the control wires 240 distally). Each of the control wires 240 may extend through a respective sheath 242, such as the above-described sheath 142. The various details and use and operation of the control wires 240 and the tissue-separating system 200 illustrated in FIG. 3 and FIG. 4 may be substantially similar to the use and operation of the control wires 140 and the tissue-separating system 100 illustrated in FIG. 1, FIG. 1A, and FIG. 2 and described above. Accordingly, for the sake of brevity, and without intent to limit, reference is made to the above descriptions as applicable, mutatis mutandis, to the control wires 240 and sheaths 242 and tissue-separating system 200 illustrated in FIG. 3 and FIG. 4. As may be appreciated, the provision of more than two tissue separator arms 220 and associated control wires 240 may provide increased options for articulation of the tissue separator device 210 illustrated in FIG. 3 and FIG. 4 in comparison with the tissue separator device 110 of FIG. 1, FIG. 1A, and FIG. 2.

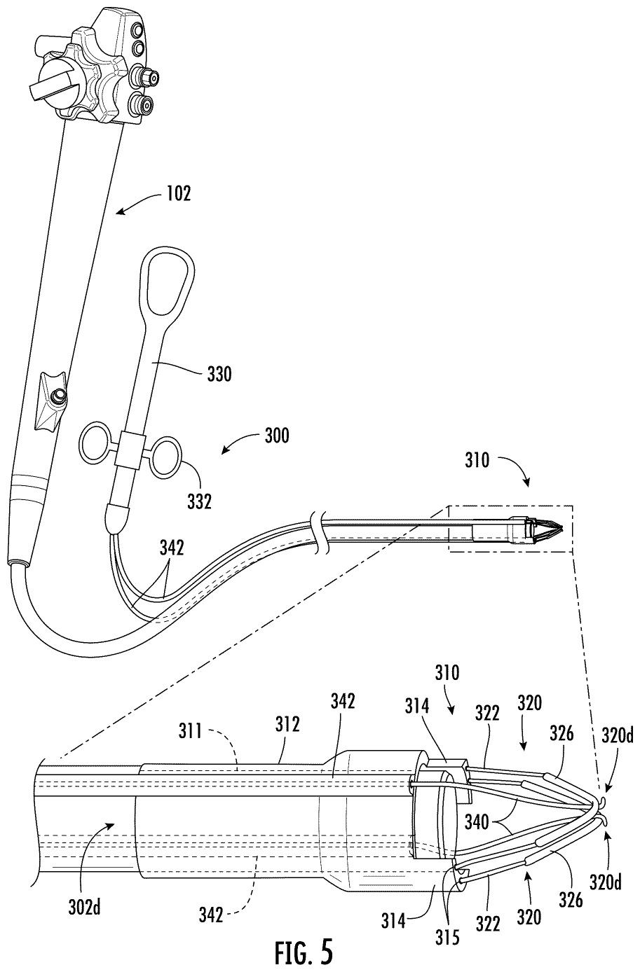

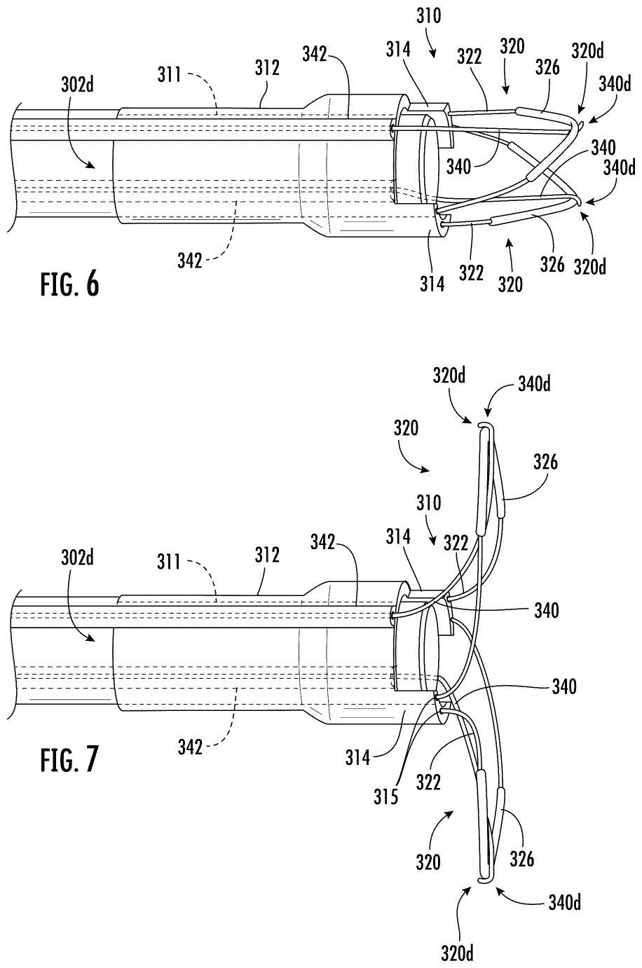

The tissue separator arms 120 and 220 illustrated in FIGS. 1-4 may be considered to have a solid or continuous external surface area available to contact tissue. In some embodiments, a fully continuous external surface area need not be provided for contact with tissue to be separated. For instance, in the example of an embodiment of a tissue-separating system 300 illustrated in FIGS. 5-7, the tissue separator device 310 has tissue separator arms 320 which present a peripheral frame or skeleton with empty space across the width of the tissue separator arms 320 without material extending across the full width or length of the tissue separator arms 320. For instance, the tissue separator arms 320 of the example of an embodiment of a tissue separator device 310 illustrated in FIGS. 5-7 may be formed from wires 322. The wires 322 may be formed from a biocompatible metal such as Nitinol, with sufficient rigidity to penetrate into tissue and to move tissue as the tissue separator arms 320 are shifted from a closed configuration (such as illustrated in FIG. 5) towards an open configuration (such as illustrated in FIG. 6 and FIG. 7). The distal ends 320d of the tissue separator arms 320 may include a tissue engagement feature 324 such as the above-described tissue engagement feature 124, reference being made to the description thereof as applicable to the tissue engagement features 324 for the sake of brevity, and without intent to limit. In some aspects, the distal ends 320d of the tissue separator arms 320 are thicker than the more proximal portions of the tissue separator arms 320. In some aspects, a hypotube 326 may be provided or extended across the distal ends 320d of the tissue separator arms 320 to impart greater strength and rigidity, such as to improve the ability of the tissue separator arms 320 to penetrate into tissue when the tissue-separating system 300 is advanced distally into tissue. One or more tissue engagement features 324 (e.g., spaced apart from one another) may be provided on the hypotube 326. The wires 322 forming the tissue separator arms 320 may be shaped to define a blunt distal end 320d of the tissue separator arms 320. In some aspects, the distal ends 320d may be tapered to facilitate insertion into tissue, such as into an initial small incision in tissue (such as described above). The thickness of the wires 322 is sufficient to not cut through (not “cheesewire”) through tissue.

The tissue separator device 310 includes a proximal mounting portion 312 defining an opening or counterbore 311 sized, shaped, configured, and/or dimensioned to receive, without impeding or obstructing the various functionalities of, the distal end 302d of a medical delivery device 302 (e.g., similar to the mounting of the above-described proximal mounting portion 112, reference being made thereto, for the sake of brevity and without intent to limit, as applicable mutatis mutandis). The proximal mounting portion 312 may be in the form of a collar or end cap with mounts 314 for the ends of the wires 322. For instance, in the example of an embodiment illustrated in FIGS. 5-7, a pair of mounts 314 extend distally from the proximal mounting portion 312. Each mount 314 includes a pair of bores 315 to receive, and to secure with respect to the proximal mounting portion 312, an end of each of the wires 322 forming the tissue separator arms 320. Although the mounts 314 are illustrated diametrically opposed from each other, the present disclosure need not be so limited and various modifications may be made to modify the spacing of the tissue separator arms 320.

Actuation of the tissue separator arms 320 may be effected in any of a variety of manners known to those of ordinary skill in the art. In the example of an embodiment illustrated in FIGS. 5-7, a control handle 330 similar to the above-described control handle 130 is provided at the proximal end 300p of the tissue-separating system 300. Like the control wire 140 and control handle 130 of the above-described tissue-separating system 100, a proximal end 340p of a control wire 340 may be operably coupled to an actuator 332 of the control handle 330 and a distal end 340d of the control wire 340 may be fixed with respect to a distal end 320d of a tissue separator arm 320. In some aspects, each of the control wires 340 may extend through a respective sheath 342, such as the above-described sheath 142 (reference being made to the above descriptions as applicable, mutatis mutandis, to the control wires 340 and sheaths 342 illustrated in FIGS. 5-7). In some aspects, the control wires 340 exit the respective sheaths 342 to be operably coupled with a respective tissue-separator arm 320 (such as to a distal end 320d of a respective tissue separator arm 320). Longitudinal sliding of the actuator 332 with respect to the control handle 330 proximally translates the control wire 340 to shift the tissue separator arms 320 between a closed configuration and an open, tissue-separating configuration For instance, a control wire 340 may extend from the control handle 330 to the distal end 320d of each of the tissue separator arms 320 such that proximal pulling on the control wires 340 proximally pulls on the distal ends 320d of the tissue separator arms 320 to shift the tissue separator arms 320 from a closed configuration, as illustrated in FIG. 5, laterally way from the longitudinal axis LA of the tissue separator device 310, such as illustrated in FIG. 6. Further proximal pulling of the control wires 340 shifts the tissue separator arms 320 further apart, as illustrated in FIG. 6 and FIG. 7, to separate tissue contacted by the tissue separator arms 320. The tissue separator arms 320 may be biased to return to a closed configuration upon release of proximal pulling force on the control wires 340 and/or may be returned to a closed configuration by distal longitudinal translation of the control wires 340 (e.g., by pushing the control wires 340 distally).

In some aspects, the tissue separator arms 320 of the example of an embodiment of a tissue separator device 310 illustrated in FIGS. 5-7 may be more flexible than the tissue separator arms 120 and 220 of the tissue separator devices 110, 210, respectively. Such flexibility may allow for a greater range of motion, such as allowing for an increase in pivoting movement and range towards an open configuration. In some aspects, the tissue separator arms 320 of a tissue separator device 310 as illustrated in FIGS. 5-7 may pivot close to 180° from their initial starting point, such as illustrated in FIG. 7. As such, the configuration of the tissue separator arms 320 of the example of an embodiment of a tissue separator device 310 as illustrated in FIGS. 5-7 may allow for further separation apart from each other than achievable by the tissue separator arms 120 illustrated in FIG. 1, FIG. 1A, and FIG. 2, or the tissue separator arms 220 illustrated in FIG. 3 and FIG. 4.

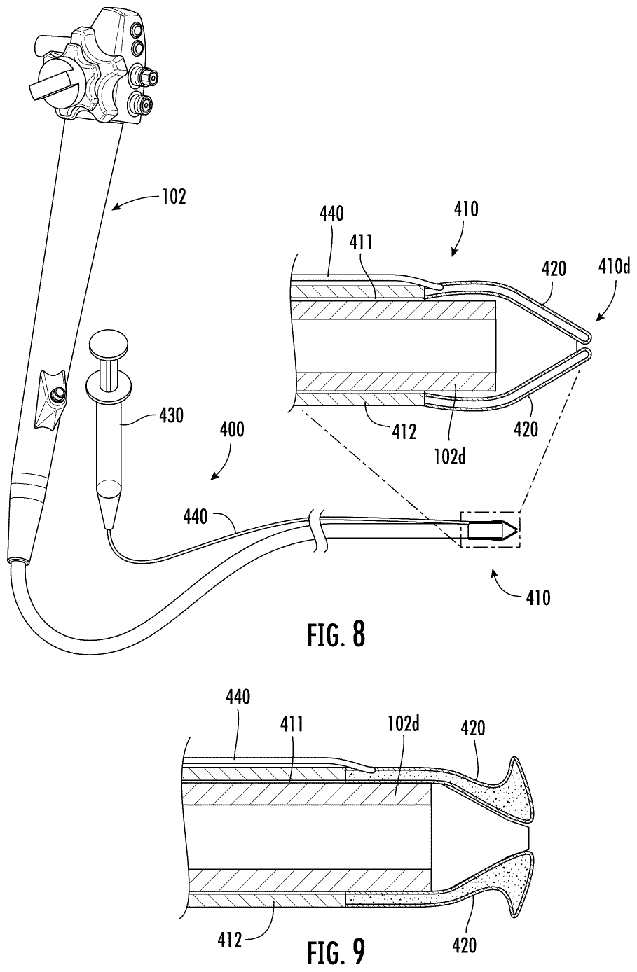

Instead of having tissue separator arms which pivot or otherwise move away from each other, a tissue separator device formed in accordance with various principles of the present disclosure may have tissue separator arms which are expandable to separate tissue contacted by the exteriors of the tissue separator arms. For instance, in the example of an embodiment illustrated in FIG. 8 and FIG. 9, a tissue-separating system 400 has a tissue separator device 410 with a proximal mounting portion 412 configured to be mounted on the distal end 102d of a medical delivery device 102, and tissue separator arms 420 formed by an inflatable element formed over and/or mounted on a distal end 412d of the proximal mounting portion 412. The proximal mounting portion 412 may be similar to the above-described proximal mounting portion 112, including a counterbore 411 sized, shaped, configured, and/or dimensioned to receive the distal end 102d of the medical delivery device 102, reference being made to the above description of the proximal mounting portion 112 for the sake of brevity and without intent to limit, as applicable mutatis mutandis.

Instead of a control wire, as in the above-described embodiments, the tissue separator device 410 of FIG. 8 and FIG. 9 uses an inflation medium (e.g., a fluid such as air or saline) to control expansion of the tissue separator arms 420 to separate tissue. As illustrated in FIG. 8, the tissue-separating system 400 includes a fill line 440, such as a fluid lumen, extending from a control handle 430 to the tissue separator arms 420. The control handle 430 is configured to supply the fill line 140 with inflation medium. In some aspects, the control handle 430 may be a syringe filled with the inflation medium. It will be appreciated that other configurations of control handles are within the scope and spirit of the present disclosure.

Once the distal end 410d of the tissue separator device 410 is inserted into an initial incision, the tissue separator arms 420 may be filled with fluid to expand and separate tissue contacted by the tissue separator arms 420. The inflatable tissue separator arms 420 may be formed of a noncompliant material to define a relatively fixed shape when in a fully inflated configuration. In some aspects, the relatively fixed shape of the noncompliant material allows laterally outward expansion of the exterior of the tissue separator arms 420 without the interior surface expanding inwardly. The tissue separator device 410 may thereby define an internal negative space between the tissue separator arms 420. Such negative space may leave an open area distal to the distal end 102d of a medical delivery device 102 coupled to the tissue separator device 410 so that devices extending from the distal end 102d of the medical delivery device 102 are capable of being used unimpeded or unobstructed by the tissue separator device 410. In some aspects, once the tissue separator arms 420 are inflated, the relatively fixed shape of the noncompliant material may form an aggressive angle extending radially outwardly from the exterior of the tissue separator arms 420 at the distal ends 420d of the tissue separator arms 420. For instance the noncompliant material may be shaped to form one or more tissue engagement features 424 sized, shaped, configured, positioned, and/or dimensioned to engage tissue. More particularly, the one or more tissue engagement features 424 may be sized, shaped, configured, positioned, and/or dimensioned to grasp and/or penetrate tissue to grip tissue and to prevent slippage of the tissue separator arms 420 with respect to the tissue. When the tissue separator arms 420 are shifted into an open configuration, the one or more tissue engagement features 424 engage and optionally penetrate into tissue, allowing the tissue separator arms 420 to clamp onto surrounding tissue to fix the tissue-separating system 400 in place with respect to tissue at the treatment site. The one or more tissue engagement features 424 may thus be considered to be anti-backout features which inhibit or prevent the tissue-separating system 400 from being pushed proximally and backed out of the treatment site during tissue separation, similar to the above-described tissue engagement features 124, 224, 324.

As will be appreciated, the tissue separator devices described herein enhance the precision and stability of tissue-separating devices which may be used in endoscopic procedures, particularly in confined spaces where tissue manipulation and scope control are crucial. Any of the above-described tissue separator devices may be used once a small incision has been formed in tissue at a target site to gain access to the third space within the body. The tissue separator device may then be advanced to the initial cut to allow the introduction of a medical scope into the target area. The size of the incision may be carefully controlled to be just large enough for the scope to enter, minimizing trauma to the surrounding tissues. The medical scope may be inserted through the small incision and may provide visual guidance for the procedure and/or may maintain an open working channel for instruments to be advanced to the target area as needed, such as to interact with and manipulate the tissue in the target area. Various of the above-described features of the above-described tissue separator devices may maintain the position of the medical scope with respect to the target area, creating a controlled space in front of the medical scope where the medical procedure may be performed. For instance, various tissue engagement features described herein provide locking for the distal end of the medical scope, such as by forming anchor points to hook into tissue. Additionally or alternatively, various of the above-described features of the above-described tissue separator devices may maintain the position of the tissue separator device to stabilize the tissue separator device's position during tissue separation. Such stabilization allows the tissue separator device to resist backing-out of the target area as the tissue separator device separates tissue. Additionally or alternatively, such stabilization facilitates advancement of the tissue separator device distally further into tissue. It will be appreciated that the tissue separator devices disclosed herein need not rely on the inherent stiffness of the medical scope to be able to move the tissue to be separated.

In some aspects, an electrosurgical knife or electrocautery device may be advanced to the target area (e.g., by the medical delivery device of the disclosed system) to allow simultaneous cutting and cauterization of tissue, effectively controlling bleeding during the procedure, enhancing the safety and efficiency of the procedure. For instance, mounting of a tissue separator device formed in accordance with various principles of the present disclosure on a distal end of a medical scope, and positioning of the tissue separator arms distal to a visualization device of the medical scope facilitates viewing of the procedure and quick responses if a bleed is observed. The various tissue separator devices described herein thus enable advanced cutting techniques, such as Endoscopic Submucosal Dissection (ESD), to be performed more readily than with prior devices, allowing careful cutting around and underneath defects or lesions in the tissue, and precise manipulation and separation of tissue layers.

In view of the above, it should be understood that the various embodiments illustrated in the figures have several separate and independent features, which each, at least alone, has unique benefits which are desirable for, yet not critical to, the presently disclosed tissue separator devices. Therefore, the various separate features described herein need not all be present in order to achieve at least some of the desired characteristics and/or benefits described herein. Only one of the various features may be present in a tissue separator device formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

It is to be understood by one of ordinary skill in the art that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. Therefore, the present invention is not limited to only the embodiments specifically described herein, and all substitutes and modifications apparent to those skilled in the art are deemed to be within the spirit, scope, and concept of the disclosure as defined by the appended claims. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A device for separating tissue and extending along a longitudinal axis from a proximal end to a distal end, said device comprising:

2. a proximal mounting portion along the proximal end of said device and defining a counterbore sized, shaped, configured, and/or dimensioned to receive a distal end of a medical delivery device;

3. a first tissue separator arm extending distally from said proximal mounting portion and movable laterally away from the longitudinal axis; and

4. a second tissue separator arm extending distally from said proximal mounting portion and movable laterally away from the longitudinal axis;

5. wherein said first tissue separator arm and said second tissue separator arm contact and separate tissue upon moving laterally away from the longitudinal axis.

6. The device of claim 1, wherein said first tissue separator arm and said second tissue separator arm are configured to move laterally away from the longitudinal axis as well as proximally to separate tissue as well as to push tissue proximally.

7. The device of claim 1, wherein said first tissue separator arm and said second tissue separator arm are pivotable with respect to said proximal mounting portion.

8. The device of claim 3, wherein said first tissue separator arm and said second tissue separator arm are capable of flexing to form a concave tissue engaging surface.

9. The device of claim 4, wherein said first tissue separator arm and said second tissue separator arm each include a plurality of axially spaced apart articulation sections configured to facilitate flexing of said first tissue separator arm and said second tissue separator arm to form concave tissue engaging surfaces.

10. The device of claim 5, further comprising a tissue engagement feature along said tissue engaging surface configured to engage tissue to prevent slippage of said first tissue separator arm and said second tissue separator arm with respect to tissue contacted by said tissue engaging surfaces.

11. The device of claim 3, wherein said first tissue separator arm and said second tissue separator arm are each formed of a section of a tubular element and are separated by slits.

12. The device of claim 3, wherein proximal regions of said first tissue separator arm and said second tissue separator arm are thinner than distal regions of said first tissue separator arm and said second tissue separator arm to allow flexing of said first tissue separator arm and said second tissue separator arm with respect to said proximal mounting portion.

13. The device of claim 3, wherein said first tissue separator arm and said second tissue separator arm are each formed of wires forming a peripheral frame without material extending across the full width of said first tissue separator arm and said second tissue separator arm.

14. The device of claim 1, wherein said first tissue separator arm and said second tissue separator arm are expandable.

15. The device of claim 10, wherein said first tissue separator arm and said second tissue separator arm are inflatable.

16. The device of claim 1, wherein said first tissue separator arm and said second tissue separator arm form a tapered distal end of said device.

17. An end cap for a medical scope having a viewing device, said end cap extending along a longitudinal axis from a proximal end to a distal end and comprising:

18. a proximal mounting portion along the proximal end of said end cap and defining a counterbore sized, shaped, configured, and/or dimensioned to receive a distal end of a medical scope;

19. a first tissue separator arm extending distally from said proximal mounting portion and movable laterally away from the longitudinal axis; and

20. a second tissue separator arm extending distally from said proximal mounting portion and movable laterally away from the longitudinal axis;

21. wherein said first tissue separator arm and said second tissue separator arm contact and separate tissue upon moving laterally away from the longitudinal axis.

22. The end cap of claim 13, wherein said first tissue separator arm and said second tissue separator arm are spaced apart within said end cap to leave a distal end of a medical scope inserted into the proximal mounting portion unimpeded during a procedure.

23. The end cap of claim 13, wherein at least one of said first tissue separator arm or said second tissue separator arm includes a tissue engagement feature configured to prevent slippage of said first tissue separator arm and said second tissue separator arm with respect to tissue contacted by said tissue engagement feature to stabilize the medical scope with respect to a target area.

24. A method of separating tissue, said method comprising:

25. creating an initial incision in tissue at a target area;

26. inserting a distal end of a tissue separator device mounted on a distal end of a medical delivery device into the incision;

27. distally advancing the distal end of the tissue separator device into the incision; and

28. moving a first tissue separator arm and a second tissue separator arm of the tissue separator device laterally apart from each other to separate tissue at the incision.

29. The method of claim 16, further comprising moving the first tissue separator arm and the second tissue separator arm proximally to push tissue proximally and to advance the medical delivery device distally into the target area.

30. The method of claim 17, further comprising engaging a tissue engagement feature extending from at least one of the first tissue separator arm or the second tissue separator arm into tissue to inhibit slippage of the tissue separator device with respect to tissue.

31. The method of claim 17, further comprising engaging a tissue engagement feature extending from at least one of the first tissue separator arm or the second tissue separator arm into tissue to stabilize the medical delivery device with respect to tissue.

32. The method of claim 16, wherein the medical delivery device is a medical scope, said method further comprising visualizing, with the medical scope, tissue being separated by the first tissue separator arm and the second tissue separator arm extending distal to the distal end of the medical scope.
